(19) United States
(12) Patent Application Publication
(10) Pub. No.: US 2026/0056213 A1
(43) Pub. Date: Feb. 26, 2026

(54) EXTRACELLULAR VESICLES-BASED BIOMARKERS FOR PANCREATIC CANCER

(71) Applicant: THE TRANSLATIONAL GENOMICS RESEARCH INSTITUTE, Phoenix, AZ (US)

(72) Inventors: Haiyong HAN, Chandler, AZ (US); Derek CRIDEBRING, Phoenix, AZ (US); Kuntal HALDER, Phoenix, AZ (US)

(21) Appl. No.: 19/110,407

(22) PCT Filed: Sep. 12, 2023
(86) PCT No.: PCT/US2023/073989

§ 371 Date: Mar. 10, 2025

Related U.S. Application Data
(60) Provisional application No. 63/405,863, filed on Sep. 12, 2022.

(51) Int. Cl. G01N 33/68; A61K 39/00; G01N 33/53

(52) U.S. Cl. CPC G01N 33/6893; A61K 39/00; G01N 33/53; G01N 2333/4704; G01N 2333/916; G01N 2800/067; G01N 2800/60; G01N 2800/7028

(57) ABSTRACT

The disclosure concerns methods and kits of diagnosis of pancreatic cancer and monitoring disease burden in patients diagnosed with pancreatic cancer, the method comprising quantitative determination of the concentration of extracellular vesicles that are positive for one, two or three markers selected from thrombospondin-2 (THBS2), alkaline phosphatase placental-like 2 (ALPPL2), and macrophage migration inhibitory factor (MIF) in the patients' fluid samples.

### RELATED APPLICATIONS

This application claims the benefit of U.S. provisional patent application 63/405,863, filed Sep. 12, 2022, titled “Extracellular Vesicles-Based Biomarkers for Pancreatic Cancer,” the entirety of the disclosure of which is hereby incorporated by this reference.

### TECHNICAL FIELD

Embodiments of the present disclosure relate to extracellular vesicle (EV) derived protein markers, THBS2, ALPPL2, and MIF, for monitoring disease burden in patients with pancreatic cancer with increased sensitivity in both CA19-9 positive and negative patients.

### BACKGROUND

Pancreatic cancer is an aggressive malignancy that often goes undiagnosed in the early stages. Non-invasive, early, and accurate diagnosis is therefore undoubtedly the “holy grail” of pancreatic cancer research. Pancreatic ductal adenocarcinoma (PDAC), in particular, is often diagnosed too late for effective therapy. PDAC is projected to become the second leading cause of cancer death in the United States by 2020. Most of the PDAC patients are diagnosed at an advanced stage of disease, and their tumors are not surgically resectable, contributing to an overall 5-year survival rate of ˜12%. The lack of early diagnostics has made it challenging to develop therapeutics to slow or reverse PDAC.

The classic strategy for early detection biomarker advancement consists of initial retrospective phases of discovery and validation with tissue samples taken from individuals diagnosed with disease, compared with controls. Currently, blood carbohydrate antigen 19-9 (CA19-9), a Sialyl-Lewis A carbohydrate antigen, is often used as a marker for monitoring disease burden in patients with pancreatic cancer. CA19-9 is a tetrasaccharide which is highly expressed on advanced adenocarcinomas (such as, colon, stomach, and pancreatic cancer). It is known to play a role in cell-to-cell recognition processes. It is also a tumor marker used detection of pancreatic cancer. Patient with PDAC are known to have an elevated level of CA19-9 (hereinafter referred to as CA19-9 secretors). About 15% of pancreatic cancer patients, however, are Lewis antigen negative, with no or low secretion of CA19-9. Thus, there remains a need for a test to monitor disease burden in pancreatic cancer patients who do not secrete CA 19-9 or do not have an elevated level of CA19-9 (hereinafter referred to as CA19-9 non-secretors).

### SUMMARY

Novel biomarkers and combinations of biomarkers for diagnosing pancreatic cancer and and monitoring disease burden are disclosed. In some aspects, the disclosure concerns methods of diagnosing pancreatic cancer in a patient. In other aspects, the disclosure concerns methods of monitoring disease burden in patients diagnosed with pancreatic cancer, including but not limited to monitoring the appearance, disappearance, regression, or progression of lesions in patients diagnosed with or being screened for pancreatic cancer. In still other aspects, the disclosure concerns methods of treating pancreatic ductal adenocarcinoma (PDAC) in a patient, including determining continuation of pancreatic cancer treatment or dosage of pancreatic cancer treatment agent. In certain implementations, the patient does not secrete CA19-9. In other implementations, the patient does secrete CA19-9.

The methods comprise quantitative determination of the amount of one, two or three markers selected from thrombospondin-2 (THBS2) positive extracellular vesicles, alkaline phosphatase placental-like 2 (ALPPL2) positive extracellular vesicles, and macrophage migration inhibitory factor (MIF) positive extracellular vesicles in serum, plasma, or pancreatic ductal fluid samples. In some embodiments, the marker comprises THBS2. In other embodiments, the marker comprises MIF. In still other embodiments, the marker comprises ALPPL2 in combination with another marker. In some embodiments, the markers comprise (a) THBS2 and ALPPL2, (b) MIF and ALPPL2, (c) MIF and THBS2, or (d) THBS2, ALPPL2, and MIF. For some embodiments, the methods further comprise correlating the amount of marker with the size of a tumor associated with pancreatic cancer.

For methods of diagnosing pancreatic cancer or monitoring a disease burden in a patient already diagnosed with pancreatic cancer, the method comprises obtaining a sample from the patient; quantitatively determining the concentration of one or more selected markers in the sample; comparing the quantitatively determined concentration of the one or more selected markers to a healthy control value or the patient's past concentration of the selected marker; and diagnosing the presence or absence of pancreatic cancer, or monitoring the disease burden in the patient based on the comparison, wherein a concentration above the healthy control value or the patient's past concentration is indicative of pancreatic cancer or a heightened disease burden. The sample obtained is selected from the group consisting of: plasma, serum, and pancreatic ductal fluid. In some embodiments, the marker or markers for comparison is selected from the group consisting of: (i) one or both of thrombospondin-2 (THBS2) positive extracellular vesicles and macrophage migration inhibitory factor (MIF) positive extracellular vesicles or (ii) of alkaline phosphatase placental-like 2 (ALPPL2) positive extracellular vesicles and THBS2 positive extracellular vesicles or (iii) a combination of ALPPL2 positive extracellular vesicles and MIF positive extracellular vesicles or (iv) a combination of ALPPL2 positive extracellular vesicles, THBS2 positive extracellular vesicles, and IF positive extracellular vesicles. Certain methods of monitoring appearance, disappearance, regression, or progression of disease in patients diagnosed with or being screened for pancreatic cancer comprise obtaining a sample from the patient; quantitatively determining the concentration of one or more selected markers in the sample; comparing the quantitatively determined concentration of the one or more selected markers to a healthy control value or the patient's past concentration of the selected marker; and monitoring appearance, disappearance, regression, or progression of disease in patients diagnosed with or being screened for pancreatic cancer based on the comparison.

A concentration above the healthy control value or the patient's past concentration is indicative of the appearance and progression of pancreatic cancer. The sample obtained is selected from the group consisting of: plasma, serum, and pancreatic ductal fluid. In some embodiments, the markers are selected from the group consisting of: (i) one or both of thrombospondin-2 (THBS2) positive extracellular vesicles and macrophage migration inhibitory factor (MIF) positive extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF positive extracellular vesicles or (iv) a combination of ALPPL2 positive extracellular vesicles, THBS2 positive extracellular vesicles, and IF positive extracellular vesicles.

For certain methods of treating pancreatic cancer (such as PDAC) in a patient, the method comprises obtaining a first sample from the patient before neoadjuvant therapy; quantitatively determining the concentration of one or more selected markers in the first sample; administering neoadjuvant therapy to the patient; and then obtaining a second sample from the patient after administration of the neoadjuvant therapy. The method further comprises quantitatively determining the concentration of the one or more selected markers in the second sample; comparing the quantitatively determined concentration of the one or more selected markers between the first sample and the second sample; and resecting the patient's pancreatic tumor when the concentration of the one or more selected markers is decreased in the second sample compared to the first sample thereby treating the patient's pancreatic ductal adenocarcinoma. In some implementations of treating pancreatic cancer, the method further comprises obtaining a third sample from the patient after resecting the patient's pancreatic tumor; quantitatively determining the concentration of the one or more selected markers in the third sample; and administering to the subject an adjuvant therapy when the concentration of the one or more selected markers is increased in the third sample compared to the second sample. The increase concentration of the one or more selected markers is indicative of heightened disease burden. The samples obtained are selected from the group consisting of: plasma, serum, and pancreatic ductal fluid. The markers are selected from the group consisting of: (i) one or both of thrombospondin-2 (THBS2) positive extracellular vesicles and macrophage migration inhibitory factor (MIF) positive extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF positive extracellular vesicles or (iv) a combination of ALPPL2 positive extracellular vesicles, THBS2 positive extracellular vesicles, and IF positive extracellular vesicles.

In some implementations, quantitative determination of the concentration of the marker positive extracellular vesicles (EVs) in the samples is performed by fluorescently tagging the marker positive EVs using antibodies to the selected markers and determining the quantity of the EVs that positive for one or more selected markers. In some aspects, the antibody used is a fluorescence labeled antibody against the selected marker. In particular implementations, quantitative determination of the concentration of the marker positive vesicles in the samples is performed using the Nanoview EV assays. In some implementations, whether the patient secretes CA19-9 is determined by an ELISA assay.

### BRIEF DESCRIPTION OF THE DRAWINGS

Implementations will hereinafter be described in conjunction with the appended and/or included DRAWINGS, where like designations denote like elements.

FIG. 1A shows biomarker positive EV concentrations in healthy controls and patients (CA19-9 non-secretors or secretors) for alkaline phosphatase placental-like 2 (ALPPL2) and thrombospondin-2 (THBS2).

FIG. 1B shows MIF positive EV concentrations in healthy controls and patients (CA19-9 non-secretors or secretors) and serum CA19-9 concentration (determined by ELISA) in patients (CA19-9 non-secretors or secretors).

FIG. 2A shows biomarker positive EV concentrations before and after treatment in CA19-9 secretors for ALPPL2 and THBS2.

FIG. 2B shows MIF positive EV concentrations and serum CA19-9 concentrations before and after treatment in CA19-9 secretors.

FIG. 3A shows biomarker positive EV concentrations in patients whose CA19-9 were normalized after treatment for ALPPL2 and THBS2.

FIG. 3B shows MIF positive EV concentrations and serum CA19-9 concentrations in patients whose CA19-9 were normalized after treatment.

FIG. 4A shows biomarker positive EV concentrations before and after treatment in CA19-9 non-secretors for ALPPL2 and THBS2.

FIG. 4B shows biomarker positive EV concentrations before and after treatment in CA19-9 non-secretors for MIF and CA19-9.

FIG. 5A shows baseline levels for dual positive EV concentrations in healthy controls and patients (CA19-9 non-secretors or secretors).

FIG. 5B shows before and after treatment for MIF and THBS2 dual positive EV concentrations in CA19-9 non-secretor or secretors).

FIG. 5C shows before and after treatment for MIF and THBS2 dual positive EV concentrations in CA19-9 non-secretor patients.

FIG. 6A shows a double combination of MIF/APPL2 dual positive EV or ALLP2/THBS2 dual positive EV are effective for monitoring disease burden in PDAC patients (CA19-9 secretors).

FIG. 6B shows a triple combination of MIF, ALPPL2 and THBS2 are effective for monitoring disease burden in PDAC patients (CA19-9 secretors).

FIG. 6C shows a combination of AlPPL2 and THBS2 is effective for monitoring disease burden in PDAC patients (CA19-9 secretors).

FIG. 7A presents correlation plots between ALPP2 maker and tumor size (RECIST) in individual patients (CA19-9 secretors).

FIG. 7B presents correlation plots between THBS2 maker and tumor size (RECIST) in individual patients (CA19-9 secretors).

FIG. 7C presents correlation plots between MIF maker and tumor size (RECIST) in individual patients (CA19-9 secretors).

FIG. 7D presents correlation plots between CA19-9 maker and tumor size (RECIST) in individual patients (CA19-9 secretors).

FIG. 8A presents correlation plots between ALPP2 maker and tumor size (RECIST) in individual patients (CA19-9 non-secretors).

FIG. 8B presents correlation plots between THBS2 maker and tumor size (RECIST) in individual patients (CA19-9 non-secretors).

FIG. 8C presents correlation plots between MIF maker and tumor size (RECIST) in individual patients (CA19-9 non-secretors).

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. For example, well-known processes and manufacturing techniques have not been described in particular detail in order to not unnecessarily obscure the disclosure. It should be noted that there are many different and alternative configurations, devices and technologies to which the disclosed technologies may be applied. The full scope of the technology disclosed herein is not limited to the examples that are described below.

This disclosure, its aspects, and implementations, are not limited to the specific package types, material types, or other system component examples, or methods disclosed herein. Many additional components, manufacturing and assembly procedures known in the art consistent with semiconductor wafer fabrication, manufacture and packaging are contemplated for use with particular implementations from this disclosure. Accordingly, for example, although particular implementations are disclosed, such implementations and implementing components may comprise any components, models, types, materials, versions, quantities, or the like as is known in the art for such systems and implementing components, consistent with the intended operation.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As used herein, the term “neoadjuvant therapy” refers to chemotherapy administered to a patient before treatment of the primary tumor with surgery and/or radiotherapy. This term is a synonym for preoperative chemotherapy. In the context of pancreatic cancer, the primary therapy is often resection of the tumor and the corresponding adjuvant therapy is chemotherapy. In the context of pancreatic cancer, the currently known optimal neoadjuvant chemotherapy utilized FOLFIRINOX (chemotherapy combination containing the drugs leucovorin calcium (folinic acid), fluorouracil, irinotecan hydrochloride, and oxaliplatin), Gemcitabine, alone or in combination of with chemotherapeutic agents.

As used herein, the term “adjuvant therapy” refers to treatment given to a patient after the main treatment to reduce the chance of cancer returning by destroying any remaining cancer cells. For patients with non-resectable tumors or have high surgery risk, the primary therapy may be chemotherapy or radiation therapy. In the context of pancreatic cancer, the primary therapy is often resection of the tumor and the corresponding adjuvant therapy is chemotherapy. For pancreatic cancer, the current adjuvant chemotherapy therapy options include, but are not limited to, FOLFIRINOX (chemotherapy combination containing the drugs leucovorin calcium (folinic acid), fluorouracil, irinotecan hydrochloride, and oxaliplatin), the combination of Gemcitabine and Nab-Paclitaxel, the combination of Gemcitabine and Capecitabine, the combination of Gemcitabine and Erlotinib, or Gemcitabine alone.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

This disclosure relates to novel biomarkers and combinations of biomarkers for diagnosing pancreatic cancer and monitoring disease burden. The biomarkers described herein are thrombospondin-2 (THBS2) positive extracellular vesicles, alkaline phosphatase placental-like 2 (ALPPL2) positive extracellular vesicles, and macrophage migration inhibitory factor (MIF) dual positive EV extracellular vesicles. ALPPL2 is a protein that is a diagnostic biomarker for pancreatic ductal adenocarcinoma that is essentially not expressed in normal tissues. THBS2 is a protein that belongs to matricellular calcium-binding glycoprotein family and was found to be a biomarker for pancreatic cancer. MIF, also known as glycosylation-inhibiting factor (GIF), L-dopachrome isomerase, or phenylpyruvate tautomerase is a protein that is encoded by the MIF gene that is a regulator of innate immunity. In particular, the methods described herein relate to the use of (i) one or both of thrombospondin-2 (THBS2) positive extracellular vesicles and macrophage migration inhibitory factor (MIF) dual positive EV extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF dual positive EV extracellular vesicles or (iv) a combination of ALPPL2 positive extracellular vesicles, THBS2 positive extracellular vesicles, and IF dual positive EV extracellular vesicles; to diagnose pancreatic cancer in a patient, monitor disease burden (for example, monitoring appearance, disappearance, regression, progression of disease) in patients diagnosed with pancreatic cancer, treat pancreatic cancer in a patient, and/or monitor treatment efficacy in patients diagnosed with pancreatic cancer. The uses include the manufacture of diagnostic kits and medicaments for pancreatic cancer. The disclosed biomarkers are useful for diagnosis, disease monitoring, and treatment monitoring of pancreatic tumors that secrete CA19-9 as well as pancreatic tumors that do not secrete CA19-9. Accordingly, the disclosed biomarkers are useful even for suspected or diagnosed pancreatic cases for which CA19-9 secretion status is unknown.

The methods of diagnosing pancreatic cancer in a patient, monitoring disease burden in patients diagnosed with pancreatic cancer, treating pancreatic cancer, and/or monitoring treatment efficacy in patients diagnosed with pancreatic cancer comprise quantitative determination of the amount of the aforementioned biomarker or biomarker combinations in serum, plasma, or pancreatic ductal fluid samples. In some implementations, quantitative determination of the concentration of the marker positive vesicles in the samples is performed by isolation of the selected marker or markers using antibodies to the selected markers and determining the quantity of the one or more selected markers that are isolated. In some aspects, the antibody used is a fluorescence labeled antibody against the selected marker. In certain embodiments the quantitative determination is performed by the Nano View EV assays. For some embodiments, the methods further comprise correlating the amount of marker with the size of a tumor associated with pancreatic cancer.

For methods of diagnosing pancreatic cancer or monitoring a disease burden in a patient already diagnosed with pancreatic cancer, the method comprises obtaining a sample from the patient; quantitatively determining the concentration of one or more selected markers in the sample; comparing the quantitatively determined concentration of EVs that are positive for the one or more selected markers to a healthy control value or the patient's past concentration of the selected marker positive EVs; and diagnosing the presence or absence of pancreatic cancer, or monitoring the disease burden in the patient based on the comparison, wherein a concentration above the healthy control value or the patient's past concentration is indicative of pancreatic cancer or a heightened disease burden. The sample obtained is selected from the group consisting of: plasma, serum, and pancreatic ductal fluid. The marker or markers for comparison is selected from the group consisting of: (i) one or both of thrombospondin-2 (THBS2) positive extracellular vesicles and macrophage migration inhibitory factor (MIF) dual positive EV extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF dual positive EV extracellular vesicles or (iv) a combination of ALPPL2 positive extracellular vesicles, THBS2 positive extracellular vesicles, and IF dual positive EV extracellular vesicles.

Certain methods of monitoring appearance, disappearance, regression, or progression of disease in patients diagnosed with or being screened for pancreatic cancer comprise obtaining a sample from the patient; quantitatively determining the concentration of EVs that are positive for one or more selected markers in the sample; comparing the quantitatively determined concentration of EVs positive for the one or more selected markers to a healthy control value or the patient's past concentration of the selected marker positive EVs; and monitoring appearance, disappearance, regression, or progression of disease in patients diagnosed with or being screened for pancreatic cancer based on the comparison.

A concentration above the healthy control value or the patient's past concentration is indicative of the appearance and progression of pancreatic cancer. The sample obtained is selected from the group consisting of: plasma, serum, and pancreatic ductal fluid. The markers are selected from the group consisting of: (i) one or both of thrombospondin-2 (THBS2) positive extracellular vesicles and macrophage migration inhibitory factor (MIF) dual positive EV extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF dual positive EV extracellular vesicles or (iv) a combination of ALPPL2 positive extracellular vesicles, THBS2 positive extracellular vesicles, and IF dual positive EV extracellular vesicles.

For certain methods of treating pancreatic cancer (such as pancreatic ductal adenocarcinoma, PDAC) in a patient, the method comprises obtaining a first sample from the patient before neoadjuvant therapy; quantitatively determining the concentration of one or more selected markers in the first sample; administering neoadjuvant therapy to the patient; and then obtaining a second sample from the patient after administration of the neoadjuvant therapy. The method further comprises quantitatively determining the concentration of EVs that are positive for the one or more selected markers in the second sample; comparing the quantitatively determined concentration of EVs positive for the one or more selected markers between the first sample and the second sample; and resecting the patient's pancreatic tumor when the concentration of EVs positive for the one or more selected markers is decreased in the second sample compared to the first sample thereby treating the patient's pancreatic ductal adenocarcinoma. In some implementations of treating pancreatic cancer, the method further comprises obtaining a third sample from the patient after resecting the patient's pancreatic tumor; quantitatively determining the concentration of EVs positive for the one or more selected markers in the third sample; and administering to the subject an adjuvant therapy when the concentration of EVs positive for the one or more selected markers is increased in the third sample compared to the second sample. The increase concentration of EVs positive for the one or more selected markers is indicative of heightened disease burden. The samples obtained are selected from the group consisting of: plasma, serum, and pancreatic ductal fluid. The markers are selected from the group consisting of: (i) one or both of thrombospondin-2 (THBS2) positive extracellular vesicles and macrophage migration inhibitory factor (MIF) dual positive EV extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF dual positive EV extracellular vesicles or (iv) a combination of ALPPL2 positive extracellular vesicles, THBS2 positive extracellular vesicles, and IF dual positive EV extracellular vesicles.

In yet other methods of treating pancreatic cancer in a patient, the method comprises obtaining a first sample from the patient; quantitatively determining the concentration of one or more selected markers in the sample, the markers being selected from the group consisting of: (i) one or both of thrombospondin-2 (THBS2) positive extracellular vesicles and macrophage migration inhibitory factor (MIF) extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF extracellular vesicles or (iv) a combination of ALPPL2 positive extracellular vesicles, THBS2 positive extracellular vesicles, and IF extracellular vesicles, comparing the quantitatively determined concentration of the one or more selected markers to a healthy control value or the patient's past concentration of the selected marker; assessing the extent of pancreatic cancer in the patient using computed tomography (CT) or ultrasound of the patient's chest, abdomen, and pelvis after neoadjuvant therapy, thereby identifying a surgery plan for the patient; and resecting the patient's pancreatic tumor. The sample obtained is selected from the group consisting of: plasma, serum, and pancreatic ductal fluid.

For the above described methods, extracellular vesicles that may be positive for THBS2, ALPPL2, and/or MIF may be isolated from serum, plasma, or pancreatic ductual fluid using methodology known in the art. In some embodiments, isolation/purification of EVs is not required to perform the ExoView assays. In certain embodiments, the sample is centrifuged for 15 min at 10,000 g. In certain embodiments, extracellular vesicles are isolated from the fluid samples by differential centrifugation following standard protocols. For example, dead cells are removed by centrifugation at 2,000×g for 10 min, while large vesicles and apoptotic bodies are removed by centrifugation at 10,000×g for 30 min. Extracellular vesicles may also be isolated from the fluid samples using commercial isolation kits. In some implementations, the fluid samples from patients may be diluted (for example, at a ratio of 1:1 to 1:100).

In a further aspect, the disclosure incudes kits for monitoring appearance, disappearance, regression, or progression of disease in patients diagnosed with or being screened for pancreatic cancer. The kits may comprise one or more of THBS2, ALLPL2, and MIF for use in testing. In some embodiments, appropriate equipment for performing test(s) and/or instructions for performing tests may be included.

The above features and disclosure will be further understood in light of the claims included below.

### Examples

### 1. Identification of Novel Pancreatic Tumor Markers

The study obtained longitudinal samples from CA19-9 secretors and non-secretors (a total of 305 samples). Alkaline phosphatase placental-like 2 (ALPPL2), thrombospondin-2 (THBS2), and macrophage migration inhibitory factor (MIF) expression in extracellular vesicles (EVs) was monitored.

The marker positive EV concentrations were determined using the ExoView R100 instrument by Unchained Labs. Briefly, serum samples were diluted 1:20 in PBS buffer and loaded to the EV-TETRA-P chips (50 μL of diluted samples to each chip) from NanoView Biosciences. the EV-TETRA-P chips contain antibodies against EV specific proteins, CD9, CD63 and CD81, which capture EVs to chips. EVs that were captured on the chips were then detected by fluorescence labeled antibodies against THSB2, ALPPL2, and MIF. The images of the fluorescently labeled EVs captured on the chips were acquired by ExoView R100. The numbers of EVs that were positive for individual protein markers or the combination of the markers were then quantified using the ExoView Analyzer software. The particle (EV) numbers reported in the figures are the average of the three captures (CD9, CD81, and CD63) in 50 nanoliter (nL) of undiluted samples.

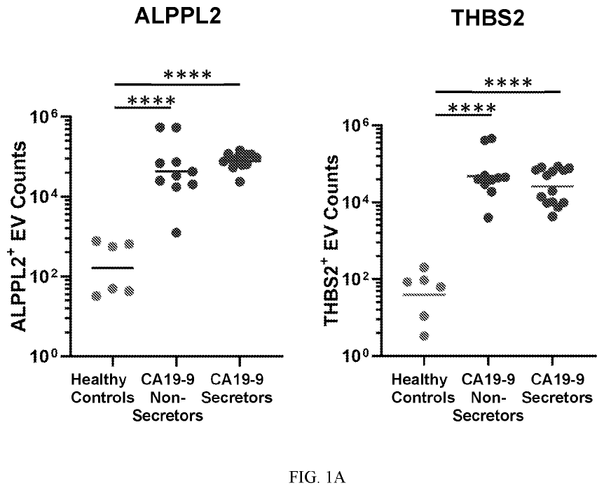

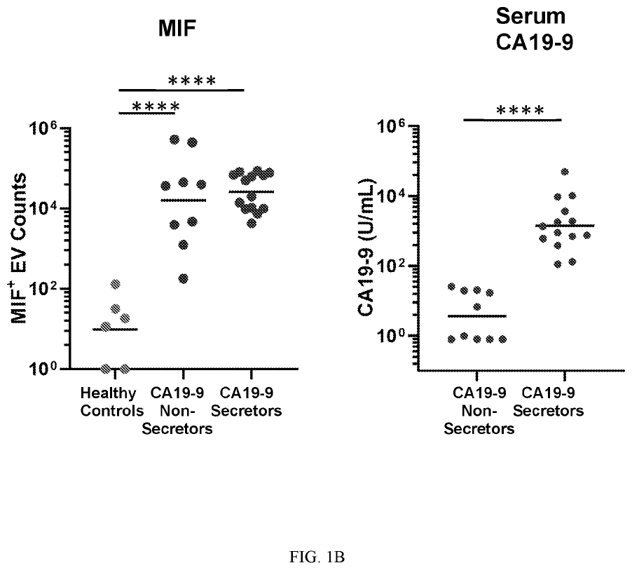

FIGS. 1A and 1B present results for biomarker positive EV concentrations in healthy controls and patients for both CA19-9 non-secretors and secretors. FIG. 1A shows biomarker positive EV concentrations in healthy controls and patients (CA19-9 non-secretors or secretors) for ALPPL2 and THBS2. FIG. 1B shows MIF positive EV concentrations in healthy controls and patients (CA19-9 non-secretors or secretors) and serum CA19-9 concentrations in patients (CA19-9 non-secretors or secretors). With ALPPL2, THBS2, and MIF, unlike CA19-9 levels, elevated levels of the marker positive EVs are found in CA19-9 secretors and non-secretors. As such elevated concentration of MIF positive EVs, ALPPL2 positive EVs, THBS2 positive EVs, in serum samples indicates the likelihood of all types of pancreatic cancer.

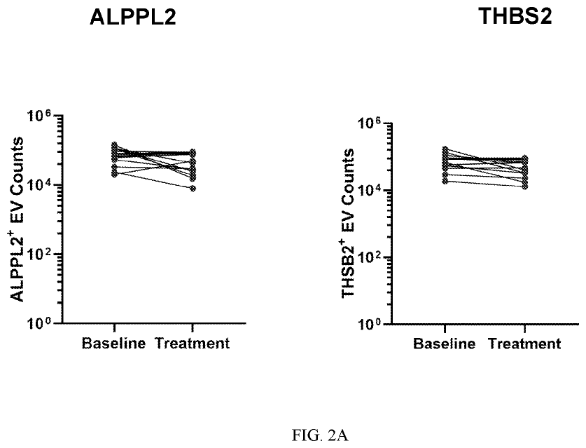

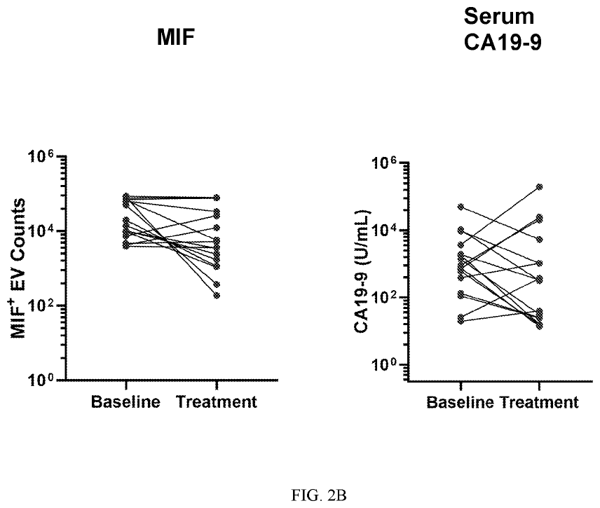

FIGS. 2A and 2B present biomarker positive EV concentrations before and after treatment in CA19-9 secretors. FIG. 2A shows biomarker positive EV concentrations before and after treatment in CA19-9 secretors for ALPPL2 and THBS2. FIG. 2B shows MIF positive EV concentrations and serum CA19-9 levels before and after treatment in CA19-9 secretors. The changes in the biomarker positive EV concentrations before and after treatment are consistent with the changes in serum CA19-9 in these patients. The top panels show the EV concentrations (Y-axis) in logarithm scale and the bottom panels show the EV concentrations in arithmetic scale.

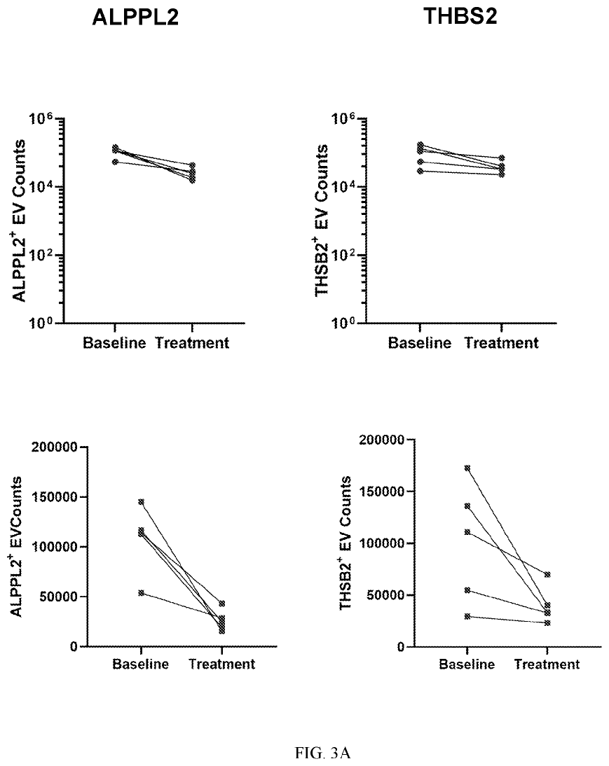

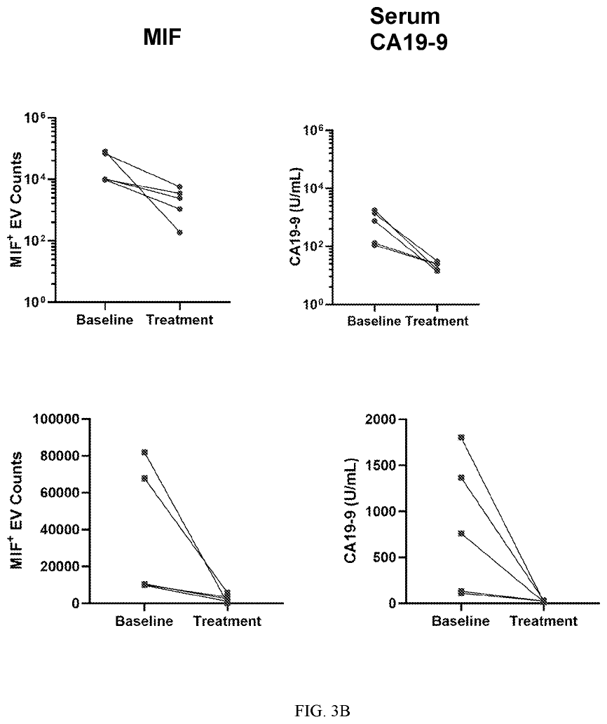

FIGS. 3A and 3B present biomarker positive EV concentrations in patients whose CA19-9 were normalized after treatment. FIG. 3A shows biomarker positive concentrations in patients whose CA19-9 were normalized after treatment for ALPPL2 and THBS2. FIG. 3B shows MIF positive EV concentrations and serum CA19-9 concentrations in patients whose CA19-9 were normalized after treatment. The changes in the biomarker positive EV concentrations before and after treatment are consistent with the changes in serum CA19-9 in these patients. The top panels show the EV concentrations (Y-axis) in logarithm scale and the bottom panels show the EV concentrations in arithmetic scale.

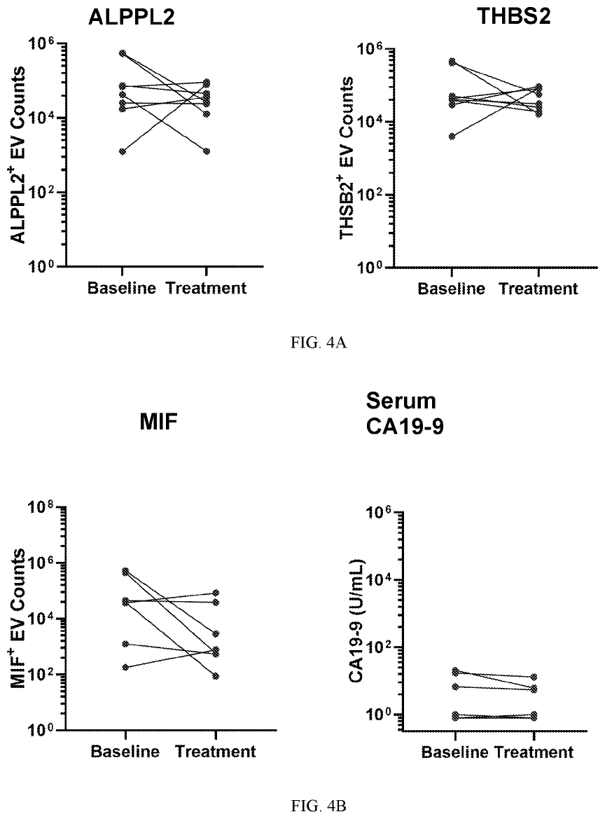

FIGS. 4A and 4B present biomarker positive EV concentrations before and after treatment in CA19-9 non-secretors. FIG. 4A shows biomarker positive EV concentrations before and after treatment in CA19-9 non-secretors for ALPPL2 and THBS2. FIG. 4B shows MIF positive EV concentrations and serum CA19-9 concentrations before and after treatment in CA19-9 non-secretors.

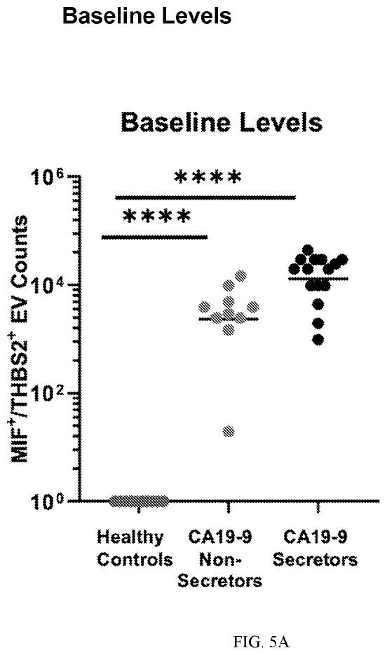

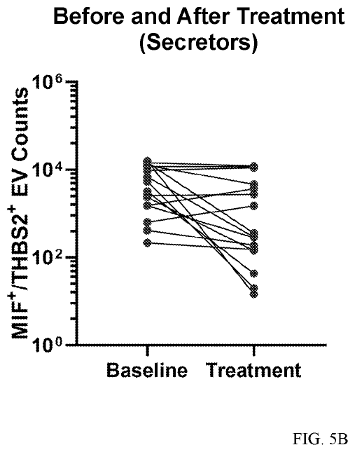

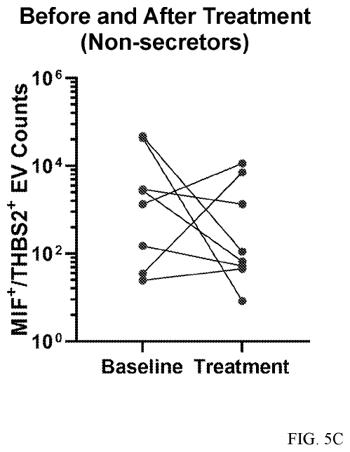

FIGS. 5A-5C present baseline and before and after treatment results for the combination THSB2 and MIF in healthy controls and both CA19-9 secretors and non-secretors. FIG. 5A shows baseline levels for THISB2/MIF dual positive EV concentrations in healthy controls and patients (CA19-9 non-secretors or secretors). FIG. 5B shows before and after treatment (secretors) for THSB2/MIF dual positive EV concentrations in patients (CA19-9 secretors). FIG. 5C shows before and after treatment (non-secretors) for THSB2/MIF dual positive EV concentrations in patients (CA19-9 non-secretors).

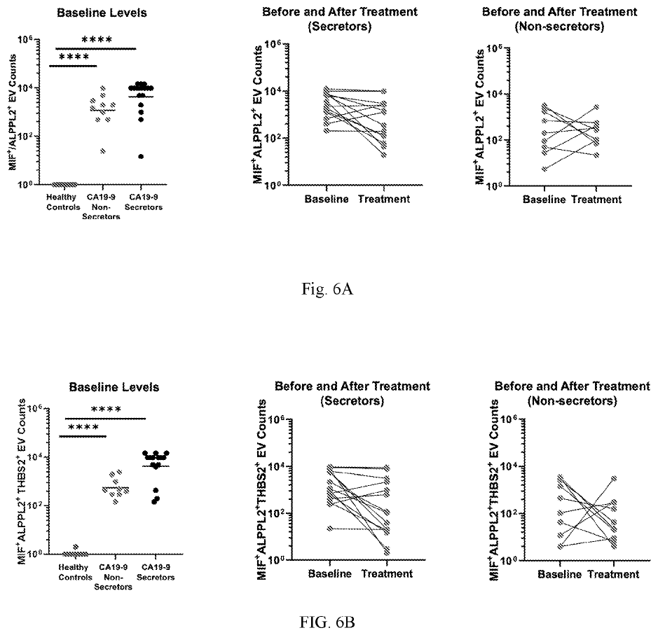

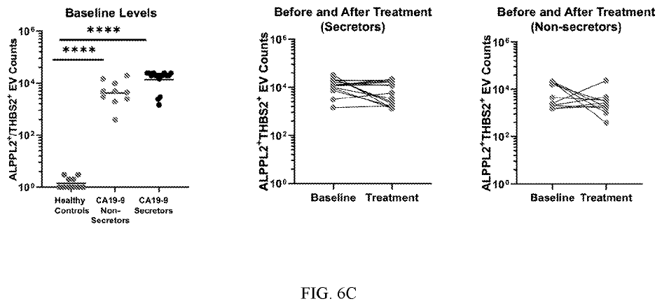

FIGS. 6A, 6B and 6C show that a combination of 2 or 3 markers can be effective for monitoring disease burden in PDAC patients. FIG. 6A shows a double combination (dual positivity) of MIF/APPL2 or ALLP2/THBS2 are effective for monitoring disease burden in PDAC patients. FIG. 6B shows a triple combination (triple positivity) of MIF, ALPPL2 and THBS2 are effective for monitoring disease burden in PDAC patients. FIG. 6C shows a double combination of AlPPL2 and THBS2 is effective for monitoring disease burden in PDAC patients (CA19-9 secretors). In some embodiments, measurements of EV are dual or triple positive for the markers as opposed to combining the measurements of a single marker positive EVs of different markers.

Accordingly, tracking the concentration of MIF positive EVs, ALPPL2 positive EVs, THBS2 positive EVs, alone or in combination in fluid samples collected from the patient is enables monitoring of disease burden and efficacy of treatment.

### 2. Correlation of Concentration of Certain EVs to Pancreatic Tumor Size

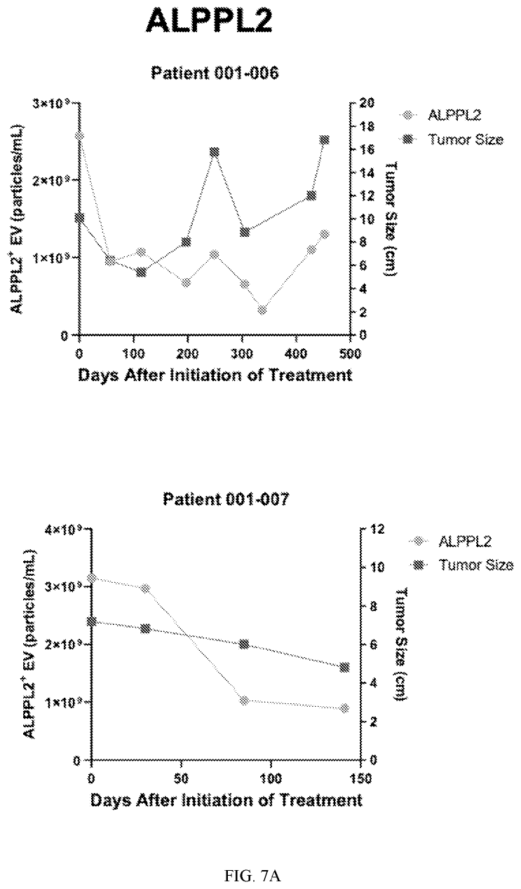

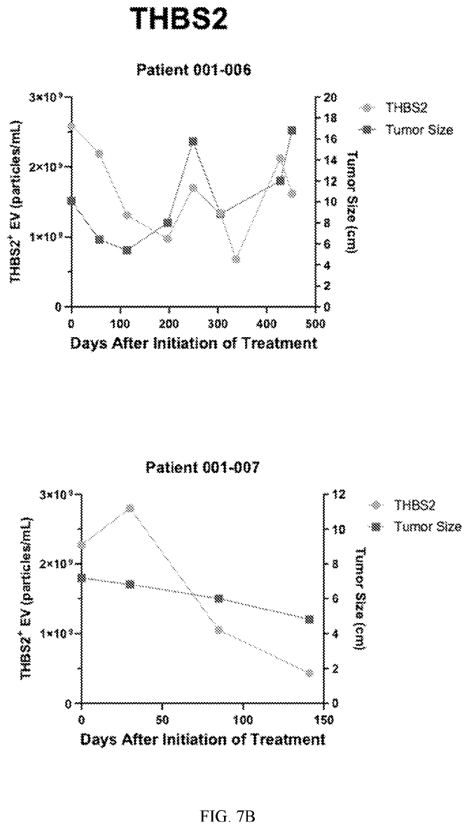

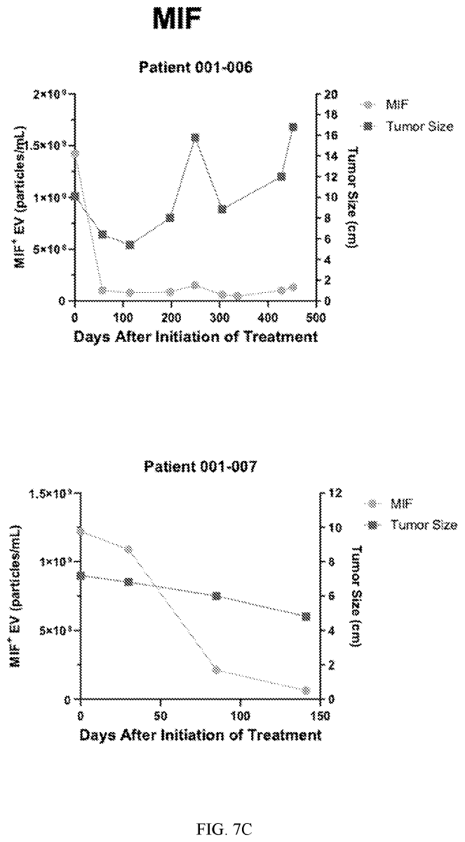

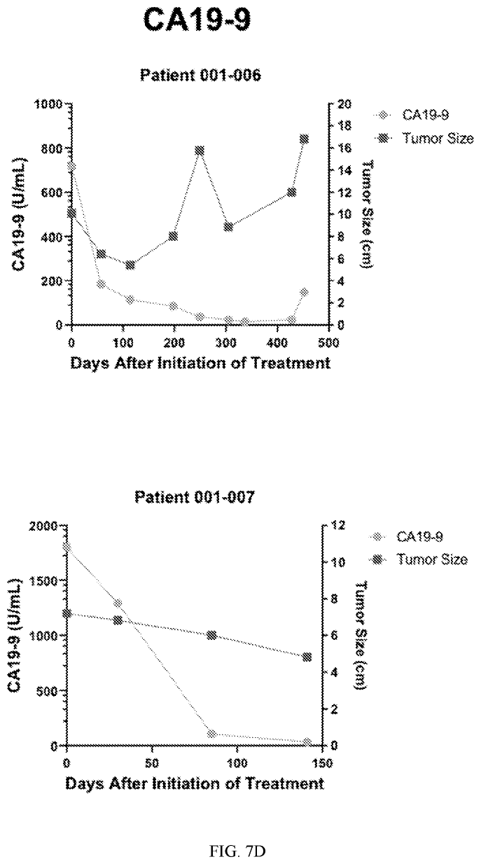

FIGS. 7A-7D presents correlation plots between various makers and tumor size, as determined by Response Evaluation Criteria in Solid Tumors (RECIST), in individual patients that were CA19-9 secretors. In these examples, the markers are ALPP2, THBS2, MIF, and CA19-9.

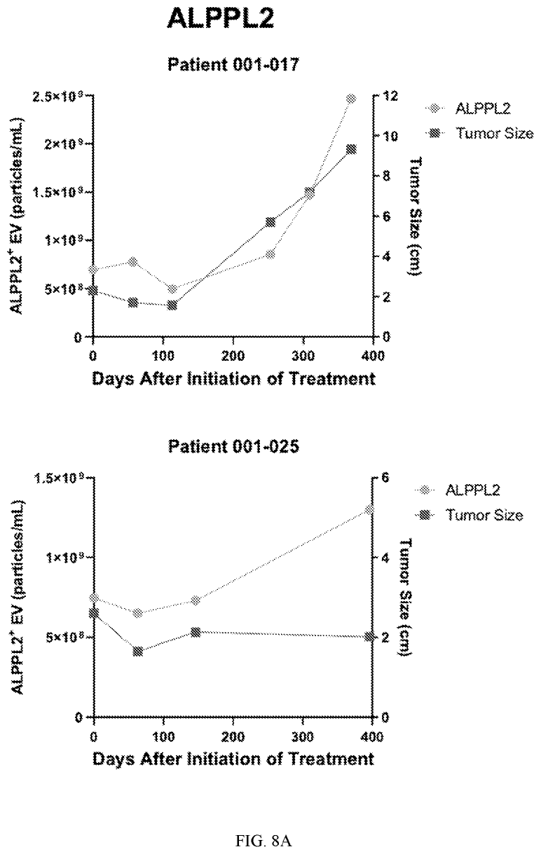

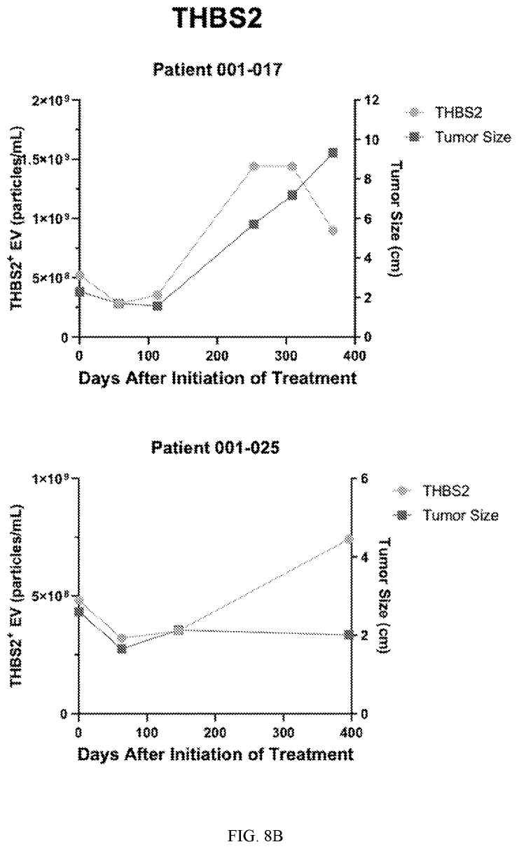

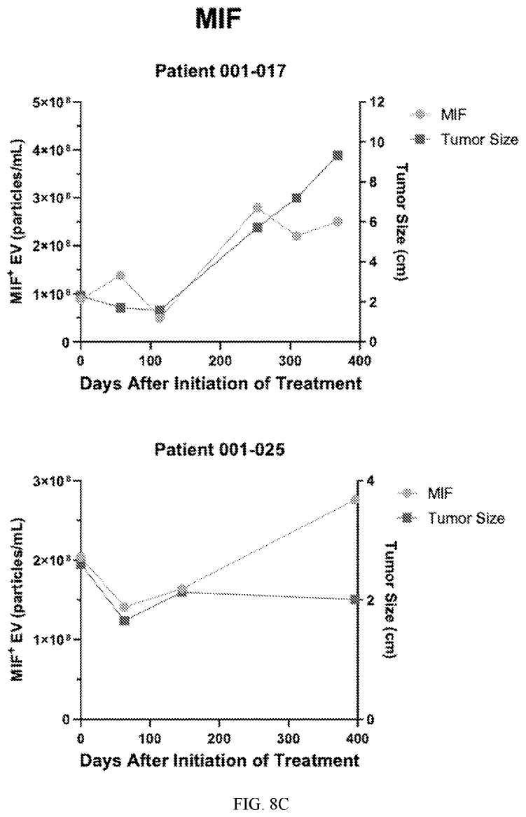

FIGS. 8A-8C presents correlation plots between maker and tumor size (RECIST) in individual patients that are CA19-9 non-secretors. The markers presented are ALPP2, THBS2, and MIF. Correlation results between EV markers and tumor size is presented in Table 1.

TABLE 1
Correlation Using Tumor Size and EV Markers

| | # of | | | | |
|---|---|---|---|---|---|
| | Patients | p Value (CD81 capture) | | | |
| Subgroup | (n) | ALPPL2 | THBS2 | MIF | CA19-9* |
| All Subjects | 26 | 0.001 | 0.001 | 0.012 | 0.057 |
| CA19-9 secretors | 16 | 0.016 | 0.014 | 0.028 | 0.045 |
| CA19-9 non-secretors | 10 | 0.003 | 0.006 | 0.120 | 0.095 |

*Data was log-transformed.

The protein-based biomarkers disclosed herein (ALLP2, THBS2, and MIF), either alone or in any combination of 2 or 3, are effective in monitoring the detection, progression, or regression of disease burden (and change in tumor size or relative size) for patients suffering from pancreatic cancer.

1. A method of diagnosing pancreatic cancer in a patient or monitoring a disease burden in a patient already diagnosed with pancreatic cancer or monitoring appearance, disappearance, regression, or progression of disease in patients diagnosed with or being screened for pancreatic cancer, the method comprising:
a) obtaining a sample from the patient, wherein the sample is selected from the group consisting of: plasma, serum, and pancreatic ductal fluid;
b) quantitatively determining the concentration of one or more selected markers in the sample, the markers being selected from the group consisting of: (i) one or both of thrombospondin-2 (THBS2) positive extracellular vesicles and macrophage migration inhibitory factor (MIF) extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF positive extracellular vesicles or (iv) a combination of ALPPL2 positive extracellular vesicles, THBS2 positive extracellular vesicles, and MIF positive extracellular vesicles;
c) comparing the quantitatively determined concentration of the one or more selected markers to a healthy control value or the patient's past concentration of the selected marker; and
d) diagnosing the presence or absence of pancreatic cancer, or monitoring the disease burden in the patient, or monitoring appearance, disappearance, regression, or progression of disease in patients diagnosed with or being screened for pancreatic cancer based on the comparison of step c), wherein a concentration above the healthy control value or the patient's past concentration is indicative of pancreatic cancer or a heightened disease burden or the appearance or progression of pancreatic cancer.

2. A method of treating pancreatic ductal adenocarcinoma in a patient, the method comprising:
a) obtaining a first sample from the patient before neoadjuvant therapy wherein the sample is selected from the group consisting of: plasma, serum, and pancreatic ductal fluid;
b) quantitatively determining the concentration of one or more selected markers in the first sample, the markers being selected from the group consisting of: (i) one or both of thrombospondin-2 (THBS2) positive extracellular vesicles and macrophage migration inhibitory factor (MIF) dual positive EV extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF dual positive EV extracellular vesicles or (iv) a combination of ALPPL2 positive extracellular vesicles, THBS2 positive extracellular vesicles, and MIF positive extracellular vesicles;
c) administering neoadjuvant therapy to the patient;
d) obtaining a second sample from the patient after administration of the neoadjuvant therapy;
e) quantitatively determining the concentration of EVs positive for the one or more selected markers in the second sample;
f) comparing the quantitatively determined concentration of EVs positive for the one or more selected markers between the first sample and the second sample; and
g) resecting the patient's pancreatic tumor when the concentration of the one or more selected markers is decreased in the second sample compared to the first sample thereby treating the patient's pancreatic ductal adenocarcinoma.

3. The method of claim 2, further comprising:
h) obtaining a third sample from the patient after resecting the patient's pancreatic tumor;
i) quantitatively determining the concentration of the one or more selected markers in the third sample; and
j) administering to the subject an adjuvant therapy when the concentration of the one or more selected markers is increased in the third sample compared to the second sample, wherein the increase concentration of the one or more selected markers is indicative of heightened disease burden.

4. The method of claim 1, further comprising treating pancreatic cancer in the patient, wherein after step (d) the method further comprises:
e) administering to the patient a neoadjuvant therapy based on step (d);
f) assessing the extent of pancreatic cancer in the patient using computed tomography (CT) or ultrasound of the patient's chest, abdomen, and pelvis after neoadjuvant therapy, thereby identifying a surgery plan for the patient; and
g) resecting the patient's pancreatic tumor.

5-9. (canceled)

10. The method of claim 1, further comprising correlating the amount of marker with a change in the size of a tumor associated with pancreatic cancer.

11. The method of claim 1, wherein the patient does not secrete CA19-9.

12. (canceled)

13. (canceled)

14. The method of claim 1, wherein determining the concentration of EVs positive for one or more selected markers is performed by detection of the selected marker or markers positive EVs using antibodies to the selected markers and determining the quantity of EVs positive for the one or more selected markers.

15. The method of claim 1, wherein the quantitative determination uses one, two, or three markers.

16. The method of claim 15, wherein the markers comprise (a) a combination of THBS2 and ALPPL2, (b) MIF and ALPPL2, or (c) MIF and THBS2.

17. The method of claim 15, wherein the markers comprise a combination of THBS2, ALLPL2, and MIF.

18. The method of claim 15, wherein the marker comprises THBS2.

19. The method of claim 15, wherein the marker comprises ALPPL2.

20. The method of claim 15, wherein the marker comprises MIF.

21-22. (canceled)

23. The method of claim 14, wherein the antibody is a fluorescence labeled antibody against the selected marker.

24. A method of determining continuation of pancreatic cancer treatment or dosage of pancreatic cancer treatment agent, the method comprising:
(a) quantitatively determining the amount of one or more markers selected from (i) one or both of thrombospondin-2 (THBS2) positive extracellular vesicles and macrophage migration inhibitory factor (MIF) positive extracellular vesicles or (ii) a combination of alkaline phosphatase placental-like 2 (ALPPL2) positive extracellular vesicles and THBS2 positive extracellular vesicles or (iii) a combination of ALPPL2 positive extracellular vesicles and MIF positive extracellular vesicles or (iv) a combination of ALPPL2 positive extracellular vesicles, THBS2 positive extracellular vesicles, and MIF positive extracellular vesicles; and
(b) correlating the amount of the one or more markers with treatment effectiveness by comparing amount of the one or more selected markers to a healthy control value or the patient's past concentration of the selected marker.

25. The method of claim 24, wherein determining the concentration of EVs positive for one or more selected markers is performed by detection of EVs positive for the selected marker or markers using antibodies to the selected markers and determining the quantity of EVs positive for the one or more selected markers.

26. The method of claim 24, wherein the one or more markers comprise (a) THBS2 and ALPPL2, (b) MIF and ALPPL2, (c) MIF and THBS2, (d) a combination of THBS2, ALLPL2, and MIF, or (e) THBS2, ALPPL2, or MIF.

27. The method of claim 24, wherein the patient does not secrete CA19-9.

28. The method of claim 25, wherein the antibody is a fluorescence labeled antibody against the selected marker.

29-30. (canceled)
